(19) United States
(12) Patent Application Publication
(10) Pub. No.: US 2015/0351610 A1
(43) Pub. Date: Dec. 10, 2015

(54) ENDOSCOPE OPERATING APPARATUS

(71) Applicant: SG ENDOSCOPY PTE. LTD., Singapore (SG)

(72) Inventor: Jin Mei Fan, Singapore (SG)

(73) Assignee: SG ENDOSCOPY PTE. LTD., Singapore (SG)

(21) Appl. No.: 14/760,788

(22) PCT Filed: Jan. 14, 2014
(86) PCT No.: PCT/SG2014/000010

§ 371 Date: Jul. 14, 2015

(30) Foreign Application Priority Data
Jan. 14, 2013 (SG) 201300286-0

(51) Int. Cl. A61B 1/005

(52) U.S. Cl. CPC A61B 1/0057; A61B 1/0052; A61B 1/0055

(57) ABSTRACT

To provide an endoscope operating apparatus where an operator who is a user can recognize a locked state and a free state clearly with his/her fingers grasping an operating part under an examining environment, and the number of constituent parts can be decreased to reduce a manufacturing cost and achieve weight reduction. An operating apparatus of an endoscope which is provided with a flexible tube inserted into a body to be examined and having a bending part at a distal end thereof and which performs work by bending the bending part according to a manual operation, comprising: a drive part which can pull a wire which is disposed in the flexible tube, a distal end of the wire being fixed to the bending part and the wire bending the bending part; and an operating part which operates the drive part.

### TECHNICAL FIELD

The present invention relates to improvement of an endoscope operating apparatus.

### BACKGROUND ART

Conventionally, endoscopes are used in a medical field or the like in order to perform examination, curing, or the like. As shown in FIG. 17, such an endoscope 200 is provided with a main body 201 and a flexible tube 203 which is joined to the main body 201, is inserted into a person to be examined, and has a bending part 202 at a distal end of the flexible tube 203, and the main body 201 is provided with an operating part 204 which can bend the bending part 202 provided at the distal end portion of the flexible tube 203 appropriately.

A wire (not shown) whose distal end is fixed to the bending part 202 and pulls the bending part 202 to bend the same is arranged within the flexible tube 203, and examination, curing and the like are performed by driving the wire in a pulling fashion by a proper means to bend the bending part 202 upward, downward, leftward or rightward. The main body 201 is provided with the operating part 204, where pulling operation of the wire is generally performed through a rotating dial 205 when operator's manual operation is performed.

In this case, when the operator has found an affected area of a body organ or the like, examination or curing is performed by putting the rotating dial 205 in a locked state to prevent the rotating dial 205 from rotating easily, and when the flexible tube 203 is pulled out of the body of the person to be operated, it is necessary to release the locked state to put the rotating dial 205 in a free state such that when the bending part 202 abuts on an inner face portion of the body organ, the bending part 202 in the bent state return to a straight state so as not to injure the inner face of the body organ.

From such a viewpoint, a lock mechanism which can switch the action of the rotating dial 205 between a locked state and a free state is generally provided in the endoscope 200. In this case, since the operator sets the locked state or the free state of the locking mechanism during examination to conduct examining work, such a locking mechanism must be configured such that the operator who is focusing on the examination can recognize the locked state or the free state clearly with feeling of his/her fingers grasping the operating part 204.

From such a viewpoint, a bending holding mechanism of an endoscope is conventionally proposed to be configured such that a leaf spring for clicking is formed in an approximately C shape, and convex portions of a concavo-convex portion are formed in the vicinity of a discontinuity portion of the approximately C shape at positions on both sides of the discontinuity portion so as to sandwich the discontinuity portion, and a plurality of projections is provided on positions on the same arc shape at equal intervals so as to face the leaf spring for clicking, so that when a bending holding operating knob is rotationally operated, two convex portions of the leaf spring for clicking get over the plurality of projections simultaneously.

In the above conventional art, however, since such a configuration is adopted that the leaf spring for clicking is formed in an approximately C shape in plan view, the convex portions are formed in the vicinity of the discontinuity portion, and these two convex portions get over four projections simultaneously, the whole configuration of the bending holding mechanism is considerably complicated and the number of parts for the bending holding mechanism is increased. Further, since the configuration is adopted where the two convex portions get over four projections simultaneously, there is such a possibility that any of the convex portions is caught by any of the projections, which results in difficulty in operation.

On the other hand, in order to avoid such a complicity that fixing operation and releasing operation of the rotating dial according to a locking operation performed by an operator are performed in an examination process in each case, an endoscope provided with an operating part having so-called auto-lock mechanism and auto-lock stopping function configured such that when an operator bends the bending part up to a predetermined state and he/she releases his/her fingers from an operating handle in the state, the operating handle is locked in the state, and when an external force acts on the bending part by contacting of the bending part which has been locked in the bent state with an inner wall of a body organ or the like, for example, at a pulling-out time of the bending part from the body of a person to be examined, the bending part returns to a straight state rapidly has also proposed.

In such an endoscope, however, since a lot of constituent parts are required, there is such a drawback that a structure becomes complicated, which results in increase of a manufacture cost. Further, since the number of constituent parts is increased and the function is complicated, a long time is required as an adjusting time of the endoscope. In addition, since the weight of the whole operating part increases according to increase of the number of constituent parts, there is such a drawback that since an operator generally performs examination while holding the operating part with his/her one hand, examining work for a long time results in his/her pain.

### DISCLOSURE OF INVENTION

### Problem to be Solved by Invention

The present invention is for solving these conventional drawbacks, and an object thereof is to provide an endoscope operating apparatus where an operator who is a user can recognize a locked state and a free state clearly with his/her fingers grasping an operating part under an examining environment, and the number of constituent parts can be decreased to reduce a manufacturing cost and achieve weight reduction.

### Means for Solving the Problem

In order to achieve the above object, according to a first aspect of the present invention, there is provided an operating apparatus of an endoscope which is provided with a flexible tube inserted into a body to be examined and having a bending part at a distal end thereof and which performs work by bending the bending part according to a manual operation, comprising: a drive part which can pull a wire which is disposed in the flexible tube, a distal end of the wire being fixed to the bending part and the wire bending the bending part; and an operating part which operates the drive part, wherein the drive part has a rack connected to the wire and a pinion gear meshing with the rack, the operating part is provided with a rotation operating mechanism which can rotationally drive the pinion gear according to manual operation of an operator, and the rotation operating mechanism has a lock mechanism which can impart braking force to a rotation operating part of the rotation operating mechanism, wherein the wire is provided with two horizontal-direction control wires for performing attitude control leftward and rightward, and two vertical-direction control wires for performing attitude control upward and downward, racks joined to the four wires, respectively, are provided, a first pinion gear meshing with the horizontal-direction control wires and a second pinion gear meshing with the vertical-direction control wires are provided, a horizontal-direction rotation operating part which can operate the first pinion gear manually and a vertical-direction rotation operating part which can operate the second pinion gear manually are provided, and wherein the lock mechanism is provided to correspond to each of the horizontal-direction rotation operating part and the vertical-direction rotation operating part.

According to a second aspect of the present invention, there is provided the operating apparatus of an endoscope according to the first aspect, wherein the lock mechanism is provided with a friction part which is disposed so as to be capable of being brought into pressure contact with the rotation operating part or being separated therefrom, and a lock operating part which can move the friction part such that the friction part is brought into contact with the rotation operating part or separated therefrom, and the rotation operating part is composed of an operating handle which rotates along an extension direction of the wire, the friction part is formed of a pad which can be brought into pressure contact with the operating handle or separated therefrom, and the lock operating part is configured so as to move the pad such that the pad is brought into pressure contact with the operating handle or separated therefrom.

According to a third aspect of the present invention, there is provided the operating apparatus of an endoscope according to the third aspect, wherein the lock mechanism is provided in the operating handle and is configured to select three positions of a lock position, a half-lock position, and a free position, and the lock operating part is provided with an operating handle disposed coaxially with the operating handle to be rotated, a position defining member formed with a position part which can select one of the three positions according to rotation of the operating handle, and an engagement part which can engage the three positions of the position defining member.

According to a fourth aspect of the present invention, there is provided the operating apparatus of an endoscope according to the fourth aspect, wherein the position defining member is formed of a cylindrical member arranged and fixed coaxially with the operating handle, recessed portions indicating the lock position, the half-lock position, and the free position are provided on a circumferential face of the cylindrical member along a circumferential direction of the cylindrical member, the operating handle is formed of a knob, and the engagement member is formed of a rod-shaped member provided on the knob and biased toward a rotation center direction of the knob.

According to a fifth aspect of the present invention, there is provided the operating apparatus of an endoscope according to the fourth aspect, wherein the knob is provided with a pressure adjusting part which adjusts biasing force of the rod-shaped member.

According to a sixth aspect of the present invention, there is provided the operating apparatus of an endoscope according to the fourth aspect, wherein the position defining member is formed of a plate-shaped member arranged and fixed coaxially with the operating handle, recessed portions indicating the lock position, the half-lock position, and the free position are provided on a flat face portion of the plate-shaped member along a rotation direction of the operating handle, the operating handle is formed as an arm part having a leaf spring part, and the engagement member is formed of a projection portion provided on the arm part.

According to a seventh aspect of the present invention, there is provided the operating apparatus of an endoscope according to the sixth aspect, wherein the arm part is provided with a pressure adjusting part which can adjust a spring pressure of the leaf spring part.

According to an eighth aspect of the present invention, there is provided the operating apparatus of an endoscope according to the second aspect, wherein the friction part is provided with a friction pad and a support part supporting the friction pad, and a braking face of the friction pad is formed with a pressure bonding preventing part which can prevent a full pressure bonding between the braking face and the rotation operating part.

According to a ninth aspect of the present invention, there is provided the operating apparatus of an endoscope according to the eighth aspect, wherein the friction pad is formed in a flat circular shape, and the pressure bonding preventing part is composed of a plurality of groove portions formed along a circumferential face direction of the friction pad.

According to a tenth aspect of the present invention, there is provided the operating apparatus of an endoscope according to the ninth aspect, wherein the friction pad is made of fluorine-containing rubber.

### Effect of Invention

According to the invention of the first aspect, since an operating apparatus of an endoscope which is provided with a flexible tube inserted into a body to be examined and having a bending part at a distal end thereof and which performs work by bending the bending part according to a manual operation, comprises: a drive part which can pull a wire which is disposed in the flexible tube, a distal end of the wire being fixed to the bending part and the wire bending the bending part; and an operating part which operates the drive part, wherein the drive part has a rack connected to the wire and a pinion gear meshing with the rack, the operating part is provided with a rotation operating mechanism which can rotationally drive the pinion gear according to manual operation of an operator, and the rotation operating mechanism has a lock mechanism which can impart braking force to a rotation operating part of the rotation operating mechanism, wherein the wire is provided with two horizontal-direction control wires for performing attitude control leftward and rightward, and two vertical-direction control wires for performing attitude control upward and downward, racks joined to the four wires, respectively, are provided, a first pinion gear meshing with the horizontal-direction control wires and a second pinion gear meshing with the vertical-direction control wires are provided, a horizontal-direction rotation operating part which can operate the first pinion gear manually and a vertical-direction rotation operating part which can operate the second pinion gear manually are provided, and wherein the lock mechanism is provided to correspond to each of the horizontal-direction rotation operating part and the vertical-direction rotation operating part, the number of constituent parts can be decreased without lowering operability and an effect of the lock mechanism, a structure can be made simple, and a manufacturing cost can be wholly reduced, which is different from a conventional endoscope provided with the operating part having so-called auto-lock mechanism and auto-lock stopping function. Further, since the weight of the whole operating part can be reduced according to decrease of the number of constituent parts, such an effect can be achieved that even if an operator performs examination while holding the operating part with his/her one hand, he/she can perform an examining work for a long time without pain.

According to the invention of the third aspect, since the lock mechanism is configured in the operating handle so as to capable of selecting three positions of the lock position, the half-lock position, and the free position, the lock operating part is provided with an operating handle disposed coaxially with the operating handle to be rotated, the position defining member formed with the position part which can select the three positions according to rotation of the operating handle, and the engagement part which can engage the three positions of the position defining member, such an effect can be achieved in the invention of the third aspect in addition to the effect of the invention of the first aspect that the operator can operate the operating handle in a semi-braking state at the half-lock position.

Therefore, when the operator advances the bending part within an internal organ in the free state to find an affected area on an inner wall of the internal organ, he/she brings the bending part close to the affected area in the semi-braking state at the half-lock position, and when the bending part reaches the affected area, he/she can perform examination or the like in the fixed state at the lock position, so that an easy-to-use endoscope which is further suitable for an actual state of the examining work can be provided.

According to the invention of the fourth aspect, the position defining member is formed of a cylindrical member arranged and fixed coaxially with the operating handle, recessed portions indicating the lock position, the half-lock position, and the free position are provided on a circumferential face of the cylindrical member along a circumferential direction of the cylindrical member, the operating handle is formed of a knob, and the engagement member is formed of a rod-shaped member provided on the knob and biased toward a rotation center direction of the knob.

Therefore, according to the invention of the fourth aspect, since the rod-shaped member biased toward the rotation center direction is caused to engage the recessed portions indicating the lock position, half-lock position, and the free position by performing rotational movement of the lock operating handle formed of the knob so that a fixing operation or a releasing operation can be performed with a proper clicking feeling, an operator can recognize the lock state, the half-lock state, or the free state clearly with his/her fingers grasping the operating part in an examining environment. As a result, an endoscope operating apparatus where while a configuration of the operating apparatus is simple and the number of constituent parts is decreased, an erroneous operation during examining work can be prevented securely can be provided.

According to the invention of the fifth aspect, since the knob is provided with a pressure adjusting part which adjusts biasing force of the rod-shaped member, even after an endoscope operating apparatus has been assembled, an operation load associated with rotational operation of the lock operating handle to the lock state, the half-lock state and the free state can be adjusted to a proper state as a final adjustment appropriately. Further, an endoscope operating apparatus where a proper operation load associated with rotational operation of the lock operating handle in response to an operator's preference can be selected appropriately can be provided.

According to the invention of the sixth aspect, the position defining member is formed of a plate-shaped member arranged and fixed coaxially with the operating handle, recessed portions indicating the lock position, the half-lock position, and the free position are provided on a flat face portion of the plate-shaped member along a rotation direction of the operating handle, the operating handle is formed as an arm part having a leaf spring part, and the engagement member is formed of a projection portion provided on the leaf spring.

Therefore, according to the invention of the sixth aspect, since the projection portion formed on the leaf spring part is caused to engage recessed portions indicating the lock position, the half-lock position, and the free position formed on the position defining member formed of the plate-shaped member by rotationally moving the operating handle formed of the arm part, so that fixing operation or releasing operation can be performed with proper clicking feeling, an operator can recognize the lock state, the half-lock state or the free state clearly with his/her fingers grasping the operating part under an examining environment while a configuration of the operating apparatus is simple and the number of constituent parts thereof is decreased. As a result, an endoscope operating apparatus which can prevent an erroneous operation during examining work securely can be provided.

According to the invention of the seventh aspect, since the arm part is provided with a pressure adjusting part which can adjust a spring pressure of the leaf spring part, even after the endoscope operating apparatus has been assembled, operation loads associated with rotation operations of the lock operating handle to the lock state, the half-lock state and the free state can be adjusted to proper states appropriately. Further, since the spring pressure of the leaf spring part can be adjusted, clicking feeling suitable for operation feeling of a user can be obtained by changing and setting the spring pressure in response to use feeling of the user appropriately.

Further, according to the invention of the seventh aspect, an operating apparatus of an endoscope where even after an endoscope operation apparatus has been assembled, an adjusting work of the operation load for the lock operation can be performed, it is easy to for an operator to operate the operating apparatus, a clicking feeling is obtained securely, and examination can be performed without erroneous operation can be provided.

According to the invention of the eighth aspect, the friction part is provided with a friction pad and a support part supporting the friction pad, and a braking face of the friction pad is formed with a pressure bonding preventing part which can prevent a full pressure bonding between the braking face and the rotation operating part.

Therefore, according to the invention of the eighth aspect, since the pressure bonding preventing part is provided on the friction pad, for example, even when the friction pad is brought into pressure contact with the rotation operating part at a lock operating time and the endoscope is left for a predetermined time as it is, such an event can be prevented that the friction pad becomes difficult to be separated from the rotation operating part due to adhesion between the friction pad and the rotation operation pad when the endoscope is reused.

According to the invention of the ninth aspect, since the friction pad is formed in a flat disc shape having an opening at a central portion thereof, and the pressure bonding preventing part is composed of a spiral groove portion formed along a circumferential direction of the friction pad, the whole braking face is prevented from being pressure-bonded to the rotation operating part completely and when the pressure contact state of the friction pad to the operating handle in the lock state is released according to reverse rotation of the operating handle, stress acting on a pressure contact region formed between adjacent grooves of the groove portion can be relieved to the groove portion, so that stress deformation of the pressure contact region is easily formed and release from the pressure bonding state can be performed easily.

As a result, even when the friction pad is brought into pressure contact with the rotation operating part at a lock operating time and the endoscope is left for a predetermined time as it is, such an event can be prevented that the friction pad becomes difficult to be separated from the rotation operating part due to adhesion between the friction pad and the rotation operating part when the endoscope is reused.

According to the invention of the tenth aspect, since the friction pad is made of fluorine-containing rubber, and the fluorine-containing rubber is small in friction coefficient, is rich in abrasion resistance, and is large in load bearing, the friction pad according to the present invention develops excellent braking property.

### BRIEF DESCRIPTION OF THE DRAWINGS

FIG. 1 is a plan view showing an embodiment of an endoscope apparatus main body according to the present invention;

FIG. 2 is a side view showing the embodiment of the endoscope apparatus main body according to the present invention;

FIG. 3 is a sectional view taken along line III-III in FIG. 1 and showing an embodiment of an endoscope operating apparatus according to the present invention and showing a whole rotation operating mechanism;

FIG. 4 is an exploded perspective view showing constituent parts of the rotation operating mechanism according to a first embodiment of the endoscope operating apparatus according to the present invention;

FIGS. 5A and 5B are a plan view of a friction pad to be used and a sectional view thereof according to the first embodiment of the endoscope operating apparatus according to the present invention;

FIG. 6 is a plan view showing a structure of a lock operating part on the side of a horizontal-direction operating handle according to the first embodiment of the endoscope operating apparatus according to the present invention;

FIG. 7 is a perspective view of a position defining member used in the lock operating part on the side of the horizontal-direction operating handle according to the first embodiment of the endoscope operating apparatus according to the present invention;

FIG. 8 is a perspective view mainly showing a structure of the lock operating part on the side of a vertical-direction operating handle and the horizontal-direction operating handle according to the first embodiment of the endoscope operating apparatus according to the present invention;

FIG. 9 is a perspective view showing a position defining member used in the lock operating part on the side of the vertical-direction operating handle according to the first embodiment of the endoscope operating apparatus according to the present invention;

FIG. 10 is a perspective view showing a lock operating handle in the lock operating part on the side of the vertical-direction operating handle according to the first embodiment of the endoscope operating apparatus according to the present invention;

FIGS. 11A and 11B are a perspective view mainly showing a leaf spring pressure adjusting mechanism used in a lock operating part on the side of a vertical-direction operating handle and a perspective view showing a spring constant adjusting member used in the leaf spring pressure adjusting mechanism according to a second embodiment of the endoscope operating apparatus according to the present invention;

FIG. 12 is a perspective view showing, from a direction different from the direction shown in FIG. 11A, a whole structure of the leaf spring pressure adjusting mechanism used in the lock operating part on the side of the vertical-direction operating handle according to the second embodiment of the endoscope operating apparatus according to the present invention;

FIG. 13 is a perspective view showing a lock operating handle used in the leaf spring pressure adjusting mechanism used in the lock operating part on the side of the vertical-direction operating handle according to the second embodiment of the endoscope operating apparatus according to the present invention;

FIG. 14 is an enlarged sectional view mainly showing a structure of another aspect of the lock operating part on the side of the vertical-direction operating handle in a rotation operating mechanism according to a third embodiment of the endoscope operating apparatus according to the present invention;

FIG. 15 is a perspective view of a position defining member used in another aspect of the lock operating part on the side of the vertical-direction operating handle according to the third embodiment of the endoscope operating apparatus according to the present invention;

FIG. 16 is an enlarged perspective view conceptually-showing another aspect of the lock operating part on the side of the vertical-direction operating handle according to the third embodiment of the endoscope operating apparatus according to the present invention; and

FIG. 17 is a view generally showing a conventional endoscope apparatus.

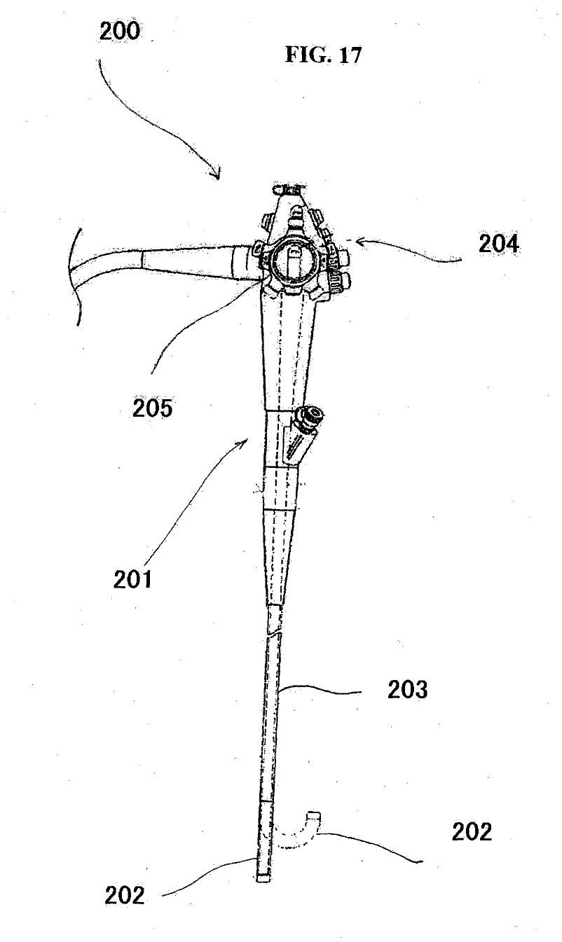

### EMBODIMENT FOR IMPLEMENTING INVENTION

The present invention will be described below in detail based upon embodiments shown in accompanying drawings.

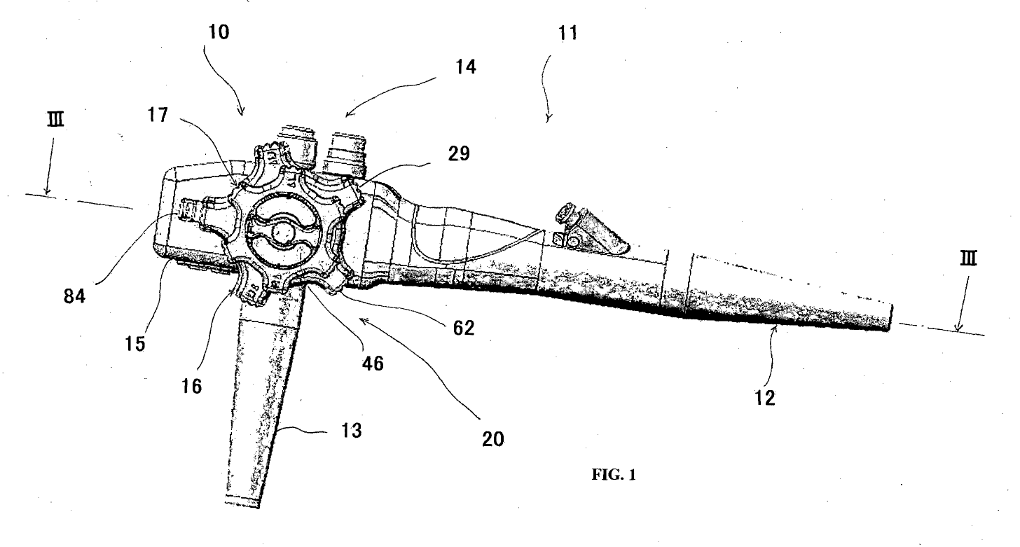

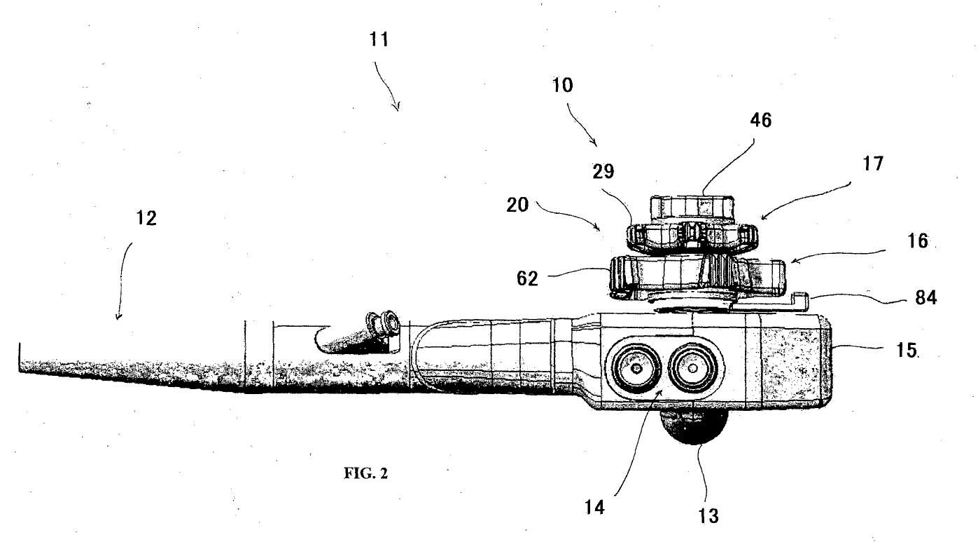

In FIG. 1 and FIG. 2, an endoscope apparatus main body 11 provided with an endoscope operating apparatus 10 according to an embodiment of the present invention is illustrated. A joint part 12 with a flexible tube is provided at a distal end portion of the endoscope apparatus main body 11, and the endoscope operating apparatus 10 is provided at a rear end portion thereof. Incidentally, in FIG. 1 and FIG. 2, reference numeral 13 denotes a connection part of a light guide cable and reference numeral 14 denotes a gas-feeding/water-feeding valve. In this embodiment, an operating part 20 composed of an vertical-direction rotation operating part 16 and a horizontal-direction rotation operating part 17 provided continuously above the vertical-direction rotation operating part 16 is provided on a body 15 constituting the endoscope apparatus main body 11.

### (Horizontal-Direction Rotation Operating Part)

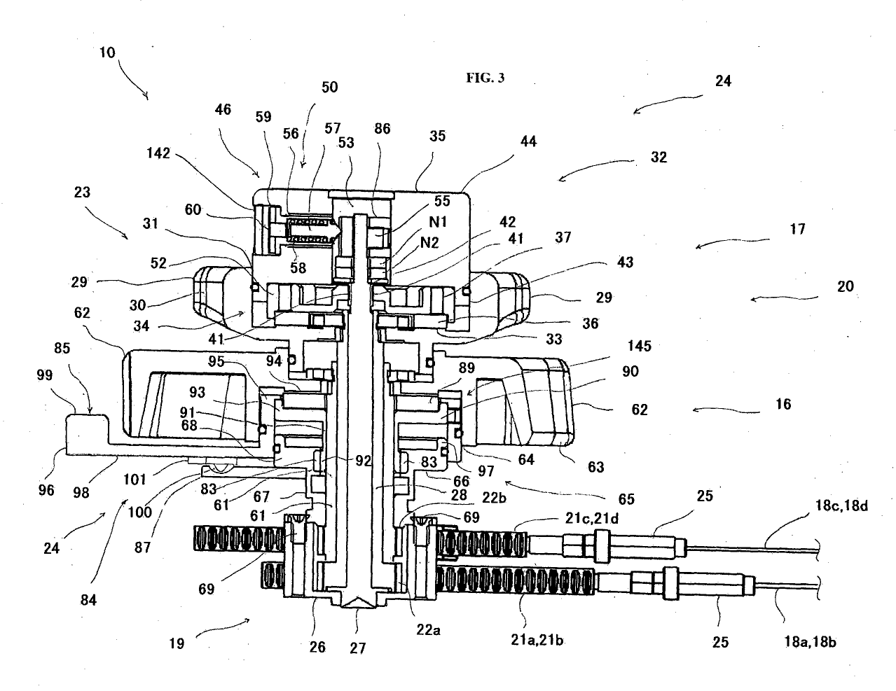

FIG. 3 is a sectional view taken along line III-III in FIG. 1 and showing the endoscope operating apparatus 10 according to the embodiment. The endoscope operating apparatus 10 according to the embodiment is provided with a drive part 19 which is configured so as to be capable of bending a bending part (not shown) provided at a distal end portion of the flexible tube by manual operation to conduct work and which can pull a wire 18 disposed within the flexible tube (not shown) and fixed to the bending part at a distal end thereof to bend the bending part by pulling the bending part, and an operating part 20 which operates the drive part 19.

The drive part 19 has a rack 21 connected to the wire 18 via an angle adjusting holder 25 and a pinion gear 22 meshing with the rack 21. The operating part 20 is provided with a rotation operating mechanism 23 which can rotationally drive the pinion gear 22 by manual operation of an operator, and the rotation operating mechanism 23 has a lock mechanism 24 which can impart braking force to the rotation operating parts 16 and 17.

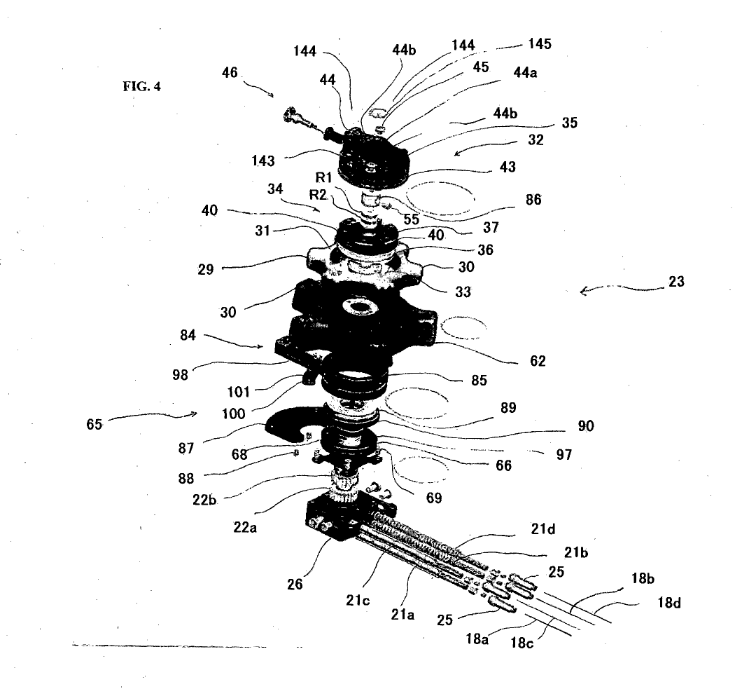

FIG. 4 is an exploded perspective view showing constituent parts of the rotation operating mechanism 23 constituting the endoscope operating apparatus 10 according to the embodiment corresponding to FIG. 3, where the wire 18 is composed of two horizontal-direction control wires 18a and 18b performing attitude control leftward and rightward and two vertical-direction control wires 18c and 18d performing attitude control upward and downward.

Horizontal-direction drive racks 21a and 21b and vertical-direction drive racks 21c and 21d arranged above the horizontal-direction drive racks 21a and 21b, which are axially joined to the four wires 18a, 18b, 18c and 18d are provided so as to extend widthwise through a gear box 26 formed in an approximately thin rectangular parallelepiped, respectively, and a first pinion gear 22a meshing with the horizontal-direction drive racks 21a and 21b and a second pinion gear 22b arranged above the first pinion gear 22a and meshing with the vertical-direction drive racks 21c and 21d are provided within the gear box 26.

Further, a horizontal-direction rotation operating part 17 which can operate the first pinion gear 22a manually and a vertical-direction rotation operating part 16 which can operate the second pinion gear 22b manually are provided above the gear box 26.

A center shaft 27 provided so as to penetrate the gear box 26 in a thickness direction thereof is fixed at a center portion of the gear box 26, and the horizontal-direction rotation operating part 17 and the vertical-direction rotation operating part 16 are assembled to the center shaft 27.

That is, a horizontal-direction drive shaft 28 having the first pinion gear 22a provided at a lower end portion thereof is disposed outside the center shaft 27 coaxially with the center shaft 27 so as to be rotatable about a circumferential face portion of the center shaft 27.

A horizontal-direction operating handle 29 is fixed to an upper end portion of the horizontal-direction drive shaft 28, and the first pinion gear 22a is rotated via the horizontal-direction drive shaft 28 by rotationally operating the horizontal-direction operating handle 29 so that the horizontal-direction drive rack 21a and 21b meshing with the first pinion gear 22a on both sides of the first pinion gear 22a in a diametrical direction thereof, respectively, can be protruded or retracted, thereby pulling the horizontal-direction control wires 18a and 18b.

As shown in FIG. 4, the horizontal-direction operating handle 29 is wholly formed in an approximately-disc shape, has a plurality of projected portions 30 formed on a circumferential edge portion thereof, and has a recessed portion 31 formed on an upper face portion thereof, and a horizontal-direction lock mechanism 32 is disposed in the recessed portion 31.

### (Horizontal-Direction Lock Mechanism)

The horizontal-direction lock mechanism 32 is provided with a friction part 34 arranged so as to be capable of coming in pressure contact with a bottom face portion 33 of the recessed portion 31 of the horizontal-direction operating handle 29 and separating therefrom, and an operating knob 35 serving as a lock operating part capable of moving the friction part 34 so as to come in pressure contact with the horizontal-direction operating handle 29 or separate therefrom.

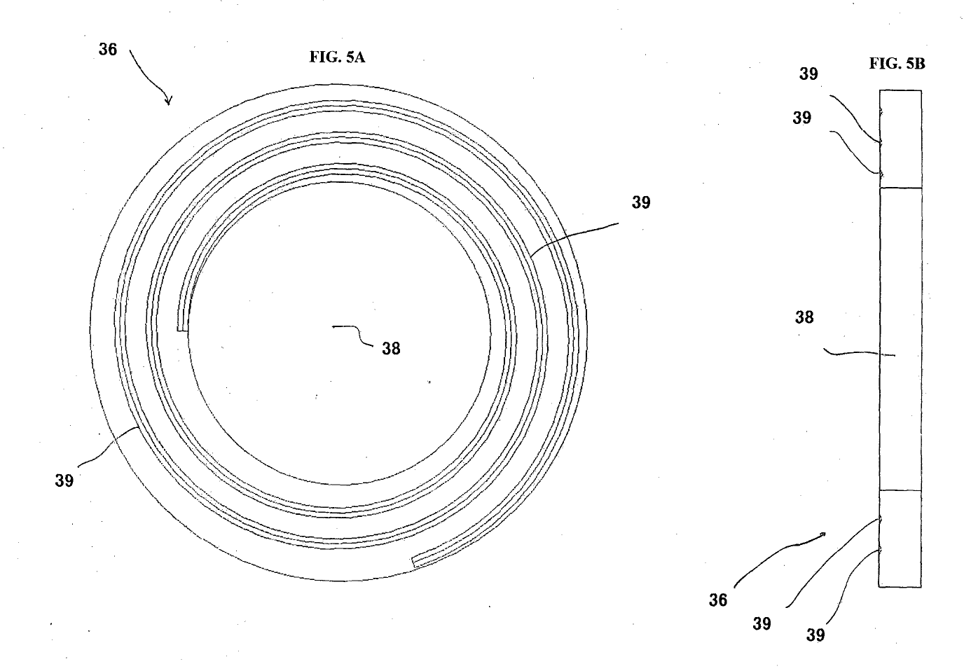

The friction part 34 is provided with a friction pad 36 and a friction pad supporting part 37 fixed with the friction pad 36. As shown in FIG. 5A and FIG. 5B, the friction pad 36 is made of fluorine-containing rubber, is formed in a flat disc shape having an opening 38 at a central portion thereof, and is provided with a pressure bonding preventing part composed of spiral groove portions 39 formed along a circumferential direction of the friction pad 36.

As shown in FIG. 4, the friction pad supporting part 37 is formed in an approximately disc shape, and the friction pad 36 is bonded and fixed to a back face portion of the friction pad supporting part 37. The friction pad supporting part 37 has a screw hole 42 provided at a central portion thereof and is rotatably engaged with and fixed to the center shaft 27 via a screw part 41 formed at an upper portion of the center shaft 27.

As shown in FIG. 4, the friction pad supporting part 37 is fixed to the operating knob 35 via a plurality of projection portions 40 formed on an upper face portion of the friction pad supporting part 37 by screws 45 and 45. As shown in FIG. 3, the operating knob 35 has a thin disc part 43 having a hollow part 52 and a knob part 44 formed on an upper face of the thin disk part 43.

As shown in FIG. 4, the knob part 44 has a rotation center part 44a formed to extend over an entire length of the thin disk part 43 in a diametrical direction and recessed portions 44b and 44b formed on both sides of the rotation center part 44a, and is configured so as to be rotationally operable by an operator with his/her fingers on the recessed portions 44b and 44b in use of the endoscope.

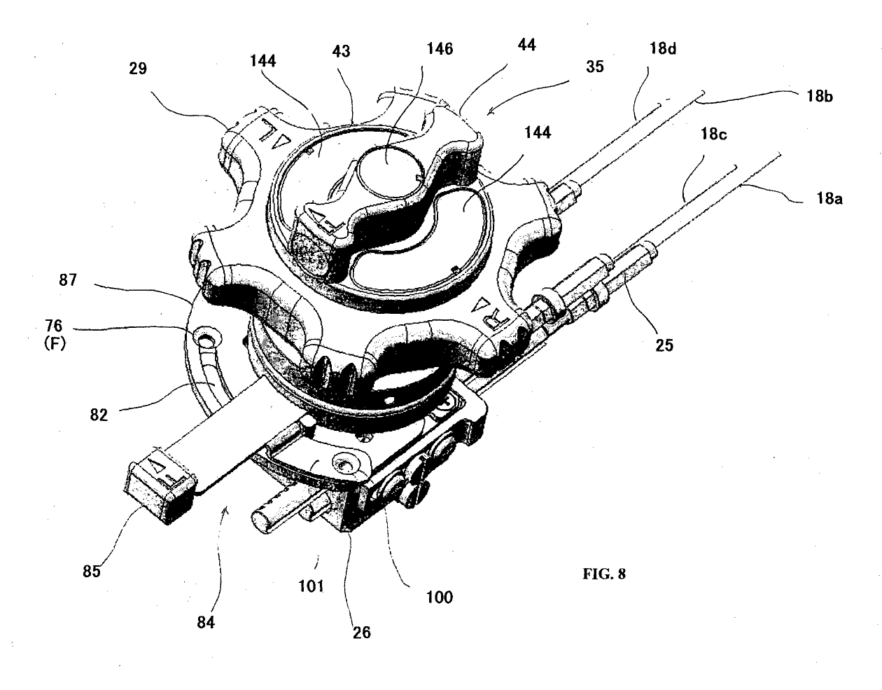

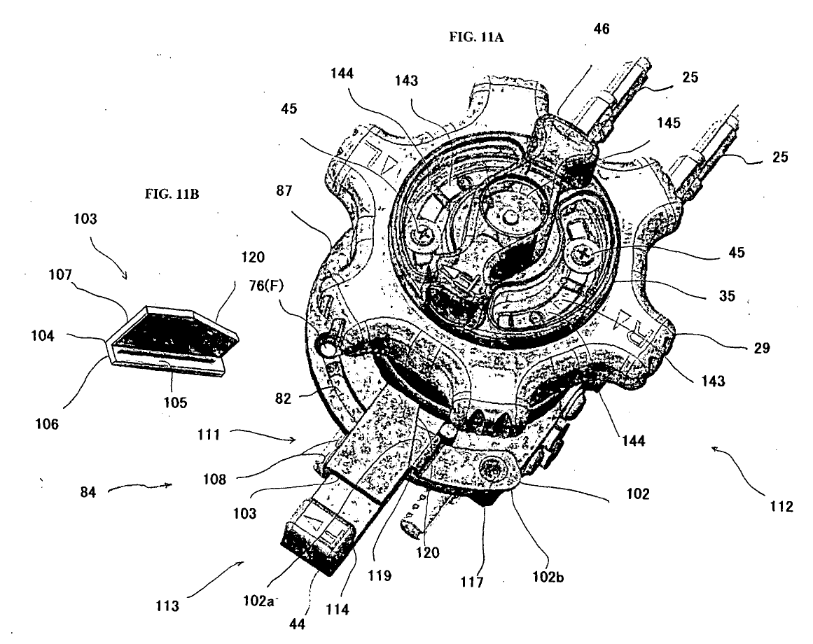

As shown in FIG. 4, FIG. 8, and FIG. 11A, curved groove portions 143 and 143 are formed on both sides of the knob part 44, and the friction pad supporting part 37 is fixed to the operating knob 35 by the screws 45 and 45 within the groove portions 143 and 143 so as to be movable along length directions of the groove portions 143 and 143. Incidentally, reference numeral 144 denotes cover members for the groove portions 143 and 143, and reference numeral 146 denotes a cover member for a cylindrical space part 53.

The friction pad supporting part 37 and the friction pad 36 are arranged and fixed within the hollow part 52, and they are configured such that a user grasps the operating knob 35 with his/her fingers to rotationally operates the same so that the friction pad supporting part 37 and the friction pad 36 are rotationally moved to be moved slightly vertically along the center shaft 27 and along the screw part 41, and the friction pad 36 can be brought into pressure contact with a bottom face portion 33 of the recessed portion 31 of the horizontal-direction operating handle 29 from the above or separated therefrom upward to brake rotation of the horizontal-direction operating handle 29 or release the braking.

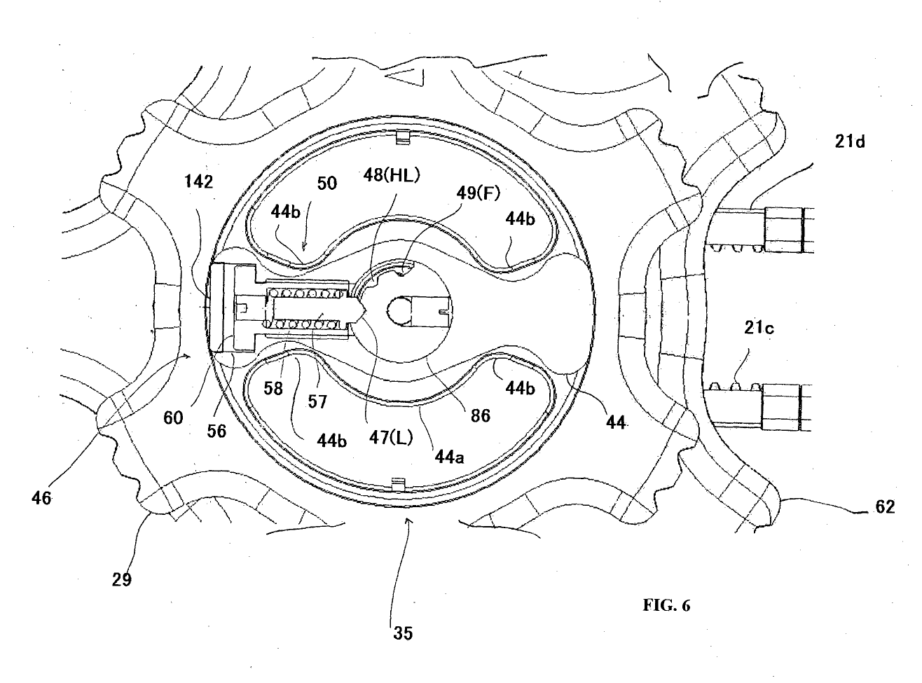

As shown in FIG. 6, a lock operating part 46 is provided within the knob part 44. The lock operating part 46 is provided with a position defining member 86 formed with a position part composed of recessed portions 47(L), 48(HL), and 49(F) by which three positions of a lock position, a half-lock position, and a free position can be selected according to rotation of the operating handle, and an engagement part 50 which can engage the three recessed portions 47(L), 48(HL), and 49(F) of the position defining member 86.

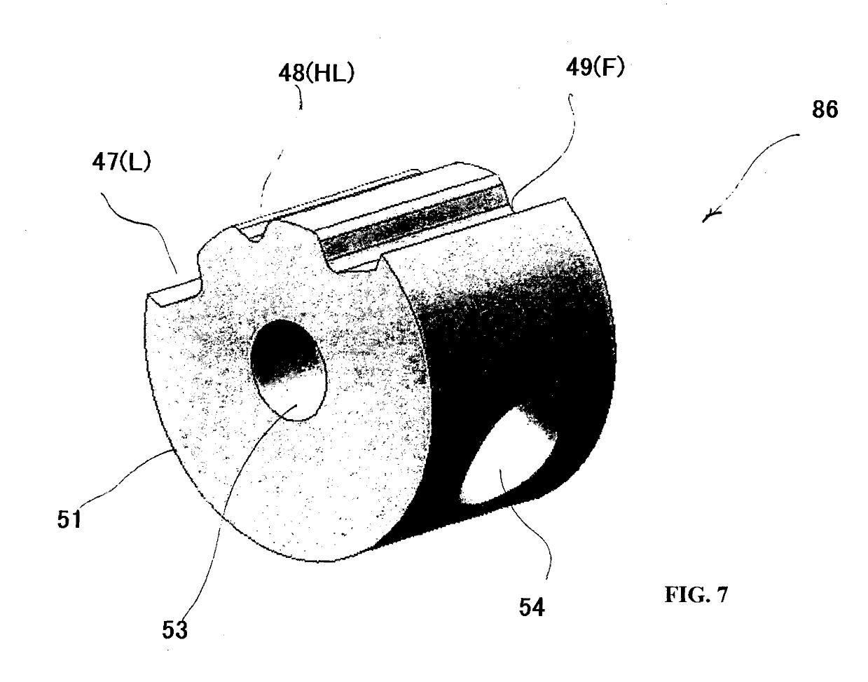

The position defining member 86 is formed of a cylindrical member 51 arranged and fixed coaxially with the horizontal-direction operating handle 29 shown in FIG. 7, and the recessed portions 47(L), 48(HL) and 49(F) corresponding to the lock position, the half-lock position, and the free position are provided on a circumferential face of the cylindrical member 51 along in a circumferential direction thereof.

As shown in FIG. 3, the cylindrical space part 53 communicating with the hollow part 52 is provided at a central portion within the operating knob 35, a distal end portion of the center shaft 27 is disposed in the cylindrical space part 53, and the position defining member 86 is disposed via nuts N1 and N2 disposed above the friction pad supporting part 37. As shown in FIG. 7, the position defining member 86 is provided with a hole 54 extending in a diametrical direction and formed with a thread groove, and it is tightened and fixed to the center shaft 27 by a screw 55.

As shown in FIG. 3 and FIG. 6, a horizontal hole 56 communicating with the cylindrical space part 53 is formed at one end portion of the knob part 44 in a lengthwise direction thereof in an intermediate portion in a height direction, and a plunger 57 and a coil spring 58 which always biases the plunger 57 in a direction of the center shaft, which constitute the engagement part 50, are housed within the horizontal hole 56. A distal end portion of the plunger 57 is formed in a taper shape, and the plunger 57 is configured so as to be capable of engaging the respective recessed portions 47(L), 48(HL) and 49(F) of the position defining member 86.

A large-diameter part 59 having a thread groove formed inside is formed at a rear end portion of the horizontal hole 56, and a screw 60 is screwed into the large-diameter part 59 to abut on a rear end portion of the plunger 57. Incidentally, reference numeral 142 denotes a cover member.

Therefore, when an operator grasps the knob 44 with his/her fingers to rotate the knob 44, he/she can select the lock position (47), the half-lock position (48) and the free position (49) appropriately by causing the plunger 57 to engage each of the recessed portions 47(L), 48(HL), and 49(F) by biasing force of the coil spring 58 and causing the plunger 57 to separate therefrom against the biasing force of the coil spring 58.

Further, in this embodiment, since the distal end portion of the screw 60 is configured so as to being capable of abutting on the rear end portion of the coil spring 58 to press the coil spring 58 in the direction of the center shaft 27, an engineer or an operator can remove the cover member 142 and can operate the screw 60 in a tightening fashion or in a loosening fashion using a driver or the like to change a spring constant of the coil spring 58 and change an engaging force of the plunger 57 to each of the recessed portions 47(L), 48(HL), and 49(F), thereby changing operation load associated with a lock operation, a half-lock operation, and a free operation in the horizontal-direction lock mechanism 32, so that even after an assembling work of the whole endoscope operating apparatus 10 has been completed, the operation load of the lock operation can be adjusted as a final adjustment or so as to suit the preferences of the operator.

### (Vertical-Direction Rotation Operating Part)

As shown in FIG. 3, a vertical-direction drive shaft 61 having the second pinion gear 22b at a lower end portion thereof is rotatably provided coaxially with the center shaft 27 outside the center shaft 27 and outside the horizontal-direction drive shaft 28 having the first pinion gear 22a at a lower end portion thereof. The second pinion gear 22b meshes with the vertical-direction drive racks 21c and 21d arranged above the horizontal-direction drive racks 21a and 21b within the gear box 26.

A vertical-direction operating handle 62 is fixed to an upper end portion of the vertical-direction drive shaft 61, and according to rotational operation of the vertical-direction operating handle 62, the second pinion gear 22b can be rotated via the vertical-direction drive shaft 61 to protrude or retreat the horizontal-direction drive racks 21c and 21d meshing with the both sides of the second pinion gear 22b in the diametrical direction, respectively, thereby pulling the vertical-direction control wires 18c and 18d. The vertical-direction operating handle 62 is arranged just below the horizontal-direction operating handle 29, has a diameter larger than the horizontal-direction operating handle 29, is wholly formed in an approximately disc shape, and has a plurality of projection portions 63 formed on a circumferential edge portion thereof.

### (Vertical-Direction Lock Mechanism)

As shown in FIG. 3, a recessed portion 64 with a flat circular shape is formed on a lower face portion of the vertical-direction operating handle 62, and a vertical-direction lock mechanism 65 is provided within the recessed portion 64.

The vertical-direction lock mechanism 65 is composed of a lock mechanism supporting member 66 and a lock operating part 84 rotatably supported by the lock mechanism supporting member 66, and the lock operating part 84 has a friction part 145, a lock operating handle 85 which can actuate the friction part 145, and a position defining member 87 provided below the lock operating handle 85 and defining a lock position, a half-lock position and a free position.

As shown in FIG. 3 and FIG. 4, the lock mechanism supporting member 66 is wholly formed in an approximately short cylindrical shape and is fixed to an upper face portion of the gear box 26. The lock mechanism supporting member 66 has a cylindrical part 67 fixed to the gear box 26 by a screw 69 and a large-diameter part 68 formed at an upper end portion of the cylindrical part 67 and having a diameter larger than the cylindrical part 67, and is disposed coaxially with the center shaft 27 outside the vertical-direction drive shaft 61.

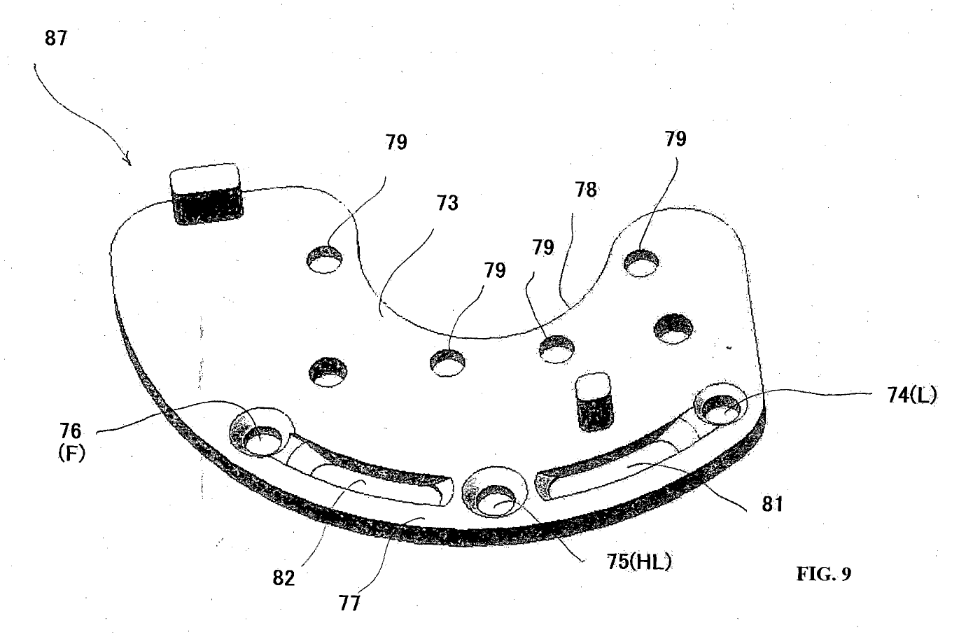

Further, a position defining member 87 is fixed to a back face portion of the large-diameter part 68 outside the cylindrical part 67. As shown in FIG. 9, the position defining member 87 is wholly formed in a curved plate shape, and is provided with a mounting part 73 formed on an inner circumferential side in a widthwise direction and a position part 77 formed in an outer circumferential side in the widthwise direction. A side edge portion 78 of the mounting part 73 is formed so as to being capable of coming in close contact with an outer circumferential face portion of the cylindrical part 67 of the lock mechanism supporting member 66, and the position defining member 87 is fixed to a back face portion of the large-diameter part 68 via a plurality of holes 79 formed inside the side edge portion 78 by screws 88.

Engagement recessed portions 74(L), 75(HL), and 76(F) indicating a lock position L, a half-lock position HL, and a free position F are provided on a surface portion of the position part 77 at predetermined intervals along an outer edge portion via groove portions 81 and 82 formed in arc shapes. The respective engagement recessed portions 74(L), 75(HL), and 76(F), and the groove portions 81 and 82 interposed therebetween are provided on the same arc line with the same curvature at the same radial distance position from an axial center of the center shaft 27. Further, screw portions 83 engaged with screw portions 92 of a friction pad supporting member 90 described later are formed in an inner peripheral portion of the large-diameter part 68 of the lock mechanism supporting member 66.

The friction part 145 is composed of a friction pad 89 and the friction pad supporting member 90 fixing and supporting the friction pad 89. The friction pad supporting member 90 is formed in a short cylindrical shape, is disposed so as to be slidable vertically relative to the vertical-direction drive shaft 61, and is provided with a shaft part 91, the screw portion 92 formed at a lower end portion of the shaft part 91 and screwed to the screw portion 83, and a supporting portion 93 of the friction pad 89 formed at an upper end portion of the shaft part 91. The supporting portion 93 is formed in a table shape and the friction pad 89 is fixed to a surface portion of the supporting portion 93.

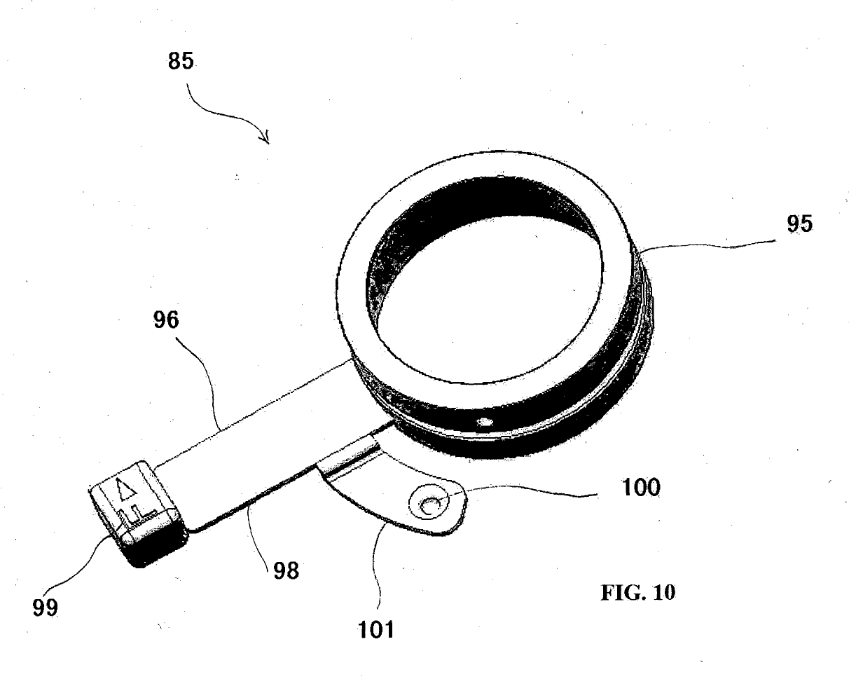

The lock operating part 84 is arranged in the recessed portion 31 formed on a lower face of the vertical-direction operating handle 62. As shown in FIG. 10, the lock operating handle 85 has a rotation part 95 formed in a short cylindrical shape and a handle main body 96 arranged so as to protrude from the rotation part 95 in a diametrical direction of the rotation part 95, and the friction part 145 is fixed inside the rotation part 95.

The supporting member 90 is fixed to an upper end of an inner circumferential face portion of the rotation part 95, the large-diameter part 68 of the lock mechanism supporting member 66 is disposed at a lower end of the inner circumferential face of the rotation part 95, and the supporting member 90 is formed to have such a diametrical size as to be capable of rotate along the large-diameter part 68 and an outer circumferential face portion of a protruded ridge portion 97 of the lock mechanism supporting member 66.

The handle main body 96 has an elongated plate-shaped arm part 98 protruded by a predetermined length diametrically outward from the rotation part 95 and a knob part 99 formed at a distal end portion of the arm part 98. A leaf spring part 101 having an engagement projection part 100 projecting in a back face direction is formed on a back face portion of the arm part 98 integrally with the arm part 98 in a region arranged above the engagement recessed portions 74(L), 75(HL), and 76(F) indicating the lock position, the half-lock position, and the free position when the lock operating handle 85 is attached to the lock mechanism supporting member 66.

As shown in FIG. 10, the lock operating handle 85 is integrally formed of such a metal as, for example, stainless steel, and the leaf spring part 101 is formed so as to extend from an upper face portion of the handle main body 96 outward in a downward-bending fashion and have a flat approximately-curved elongated plate shape, and is provided with the engagement recessed portion 100 formed at a distal end portion thereof so as to project in a back face direction.

Therefore, by rotating the handle main body 96 around the axis of the center shaft 27, the engagement projection part 100 is caused to engage the engagement recessed portions 74(L), 75(HL), and 76(F), respectively, so that the friction part 145 can be set to the lock position, the half-lock position, and the free position.

That is, when the engagement projection portion 100 is in engagement with the engagement recessed portion 74(L), the friction part 145 is located at the uppermost position where the friction pad 89 is put in a pressure contact with the lower face portion 94 of the recessed portion 64 of the vertical-direction operating handle 62, when the engagement projection portion 100 is in engagement with the engagement recessed portion 75(HL), the friction part 145 lowers slightly, where the friction pad 89 is put in a slightly abutting state on the lower face portion 94, and when the engagement projection portion 100 is in engagement with the engagement recessed portion 76(F), the friction part 145 is located at the lowermost position, the friction part 89 is completely separated from the lower face portion 94.

An operation of the endoscope operating apparatus 10 according to this embodiment will be described below.

When operation of the endoscope is performed by the endoscope operating apparatus 10 according to this embodiment, an operator inserts the flexible tube (not shown) joined and fixed to the joint part 12 of the endoscope apparatus main body part 11 into a body of a person to be examined, and when examination is performed, the operator inserts the flexible tube into a body of a person to be examined in a state where both the lock operating part 84 on the side of the vertical-direction operating handle 62 and the lock operating part 46 of the horizontal-direction operating handle 29 are put in free states.

That is, on the side of the horizontal-direction operating handle 29, an operator grasps the knob part 44 with his/her fingers to rotate the lock operating part 46 from the lock position shown in FIG. 6 in a clockwise direction in FIG. 6 to cause the plunger 57 to engage the recessed portion 49(F).

In this case, when the knob part 44 is rotated from the lock position L shown in FIG. 6 where the plunger 57 is in engagement with the recessed portion 47(L), the plunger 57 gets over the projection portion adjacent to the recessed portion 47(L) while moving outward against the biasing force of the coil spring 58, and the plunger 57 is caused to engage the adjacent recessed portion 48(HL) (half-lock position) by the biasing force of the coil spring 58.

When the knob part 44 is further rotationally operated in the clockwise direction, the plunger 57 similarly retreats against the biasing force of the coil spring 58 to get over the adjacent projection portion and then advances by the biasing force of the coil spring 58 to engage the engagement recessed portion 49(F) (the free position).

In this case, since the friction pad supporting part 37 constituting the friction part 34 rises in a direction of the distal end portion of the center shaft 27 due to engagement with the screw part 41 provided in the center shaft 27 according to rotation operation of the lock operating part 46 performed by the knob part 44, the friction pad 36 is separated from the bottom face portion 33 of the hollow part 52 of the horizontal-direction operating handle 29.

As a result, since a braking force does not acts on the horizontal-direction operating handle 29 at all, the operator can operate the horizontal-direction operating handle 29 with a very small operating force and can bend the bending part at the distal end portion of the flexible tube easily leftward and rightward. Further, when the bending part abuts on each region of internal organs, it can bend easily, so that an organ is prevented from being injured.

Therefore, according to engagement of the plunger 57 biased by the coil spring 58 to the engagement recessed portions 47(L), 48(HL), and 49(F), the operator can operate the horizontal-direction operating handle 29 from the lock position to the free position via the half-lock position with proper clicking feeling while recognizing the respective positions.

Further, the operator can perform transition from the free position to the lock position by rotating the lock operating part 46 in a counterclockwise direction in the same manner as the above.

On the other hand, on the side of the vertical-direction operating handle 62, the operator rotates the lock operating handle 85 constituting the lock operating part 84 shown in FIG. 8 from the lock position shown in FIG. 8 in the clockwise direction in FIG. 8 to cause the engagement projection portion 100 to engage the engagement recessed portion 76(F) (the free position) via the engagement recessed portion 75(HL) forming the half-lock position of the position defining member 87 shown in FIG. 9.

In this case, since the leaf spring part 101 formed with the engagement projection portion 100 is always put in a biased state to the position defining member 87, the operator, with proper clicking feeling, separates the engagement projection portion 100 from the engagement recessed portion 74(L) (the lock position) to cause the engagement projection portion 100 to engage the engagement recessed portion 74(HL) (the half-lock position) via the groove portion 81 and then cause the same to engage the engagement recessed portion 76(F) (the free position) via the groove portion 82.

Therefore, according to rotational operation of the lock operating part 84 performed by the lock operating handle 85, the friction pad supporting member 90 constituting the lock operating part 84 lowers toward the proximal end portion of the center shaft 27 due to engagement between the screw portion 83 provided in the lock mechanism supporting member 66 and the screw portion 92 of the friction pad supporting member 90, so that the friction pad 89 is separated from the lower face portion 94 of the recessed portion 64 of the vertical-direction operating handle 62.

As a result, since a braking force does not acts on the vertical-direction operating handle 62 at all, the operator can rotationally operate the vertical-direction operating handle 62 with a very small operating force and can bend the bending part at the distal end portion of the flexible tube easily upward and downward, and the operator can bend the bending part at the distal end portion of the flexible tube leftward and rightward with a very small operating force. Further, when the bending part has abutted on each region of internal organs, it can bend easily, thereby preventing an organ from being injured.

Therefore, since the leaf spring part 101 is always biased to the position defining member 87, the engagement projection portion 101 engages each of the engagement recessed portions 74(L), 75(HL), and 76(F) by the biasing force, and the operator can operate the vertical-direction operating handle 62 from the lock position to the free position via the half-lock position with proper clicking feeling while recognizing the respective positions.

Thereafter, when the operator has found a region to be fully examined or a region to be cured on an inner wall face of an organ to be examined, he/she rotates the lock operating part 46 and the lock operating part 84 in a counterclockwise direction to cause the plunger 57 of the knob part 44 to engage the recessed portion 48(HL)(the half-lock position) and cause the engagement projection portion 100 of the lock operating handle 85 to engage the engagement recessed portion 75(HL) in the same manner as the above and brings the bending part close to a target region while putting both the horizontal-direction operating handle 29 and the vertical-direction operating handle 62 in their half-lock states.

When the operator actually cuts away a region to be examined using a forceps or the like, he/she further rotates the lock operating part 46 and the lock operating part 84 in the counterclockwise direction to cause the plunger 57 of the knob part 44 to engage the recessed portion 47(L) and cause the engagement projection portion 100 of the lock operating handle 85 to engage the recessed portion 74(L) to achieve the locked state, thereby imparting large braking force to both the horizontal-direction operating handle 29 and the vertical-direction operating handle 62 such that the horizontal-direction operating handle 29 and the vertical-direction operating handle 62 do not rotate easily.

Accordingly, regarding operations of the lock operating parts 46 and 86, the operator can perform operations from the free state to the half-lock state and the lock state while recognizing the free position, the half-lock position, the lock position with clear clicking feeling, and he/she can form the free state, the half-lock state, and the lock state easily and rapidly, which results in excellent operability.

Further, regarding the lock operating handle 85 according to this embodiment, since the leaf spring part 101 having the engagement projection portion 100 projecting in the back face direction and the arm part 98 are integrally formed from metal such as stainless steel, and they are formed in elongated plate shapes having predetermined sizes, for example, when the engagement projection portion 100 is located at the lock position in such a state that it has engaged the engagement recessed portion 74 and the bending part of the flexible tube in its lock state is pulled out from an internal organ of a person to be examined, and the operator has realized that there is such a risk that the bending part abuts an inner wall of the internal organ, he/she lifts up the knob part 99 upward with his/her fingers to slightly bend the arm part 98 upward and causes the engagement projection portion 100 to engage the engagement recessed portion 76(F) which is the free position from the engagement recessed portion 74(L) without engagement with the engagement recessed portion 75(HL) at the half-lock position at once, which can result in avoidance of the risk.

FIG. 11 to FIG. 13 show a second embodiment of the endoscope operating apparatus 10 according to the present invention.

An endoscope operating apparatus 112 according to the second embodiment is different from the endoscope operating apparatus 10 according to the first embodiment in configuration of the lock operating part 84 on the side of the vertical-direction operating handle. Incidentally, same members as those of the first embodiment are attached with same reference numerals, and explanation thereof is omitted.

In the second embodiment, a leaf spring pressure adjusting mechanism 111 configured such that a pressure contacting force of an engagement projection portion 117 to the position defining member 87 can be adjusted by changing a spring constant of a leaf spring member 102 provided in a lock operating handle 113 appropriately.

The leaf spring pressure adjusting mechanism 111 is composed of the leaf spring member 102 which is constituted as a member separated from an arm part 114, which is different from the first embodiment, and whose proximal end portion is fixed to an upper face of the arm part 114 along a widthwise direction by screws (not shown), and a spring constant adjusting member 103 covering about a half of the leaf spring member 102 and arranged on an upper face portion of the arm part 114 so as to be movable along the lengthwise direction of the arm part 114.

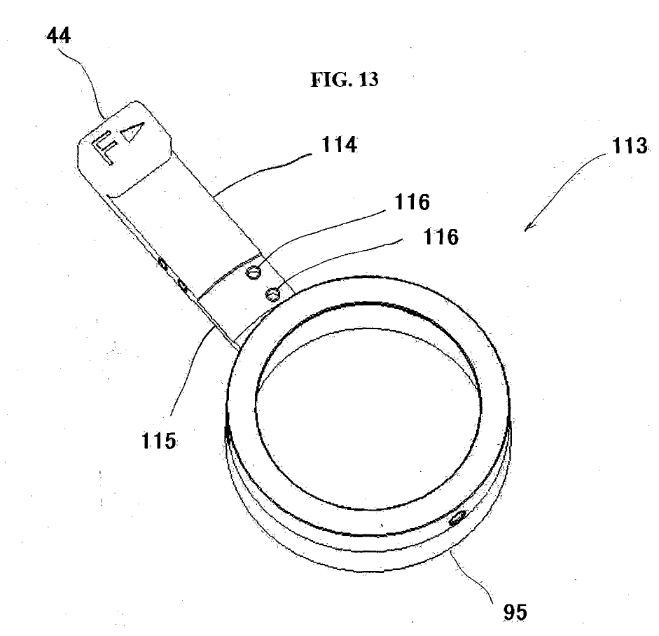

As shown in FIG. 13, in the lock operating handle 113 according to this embodiment, a leaf spring member mounting part 115 formed in a flat rectangular shape recessed from an upper face of the arm part 114 over a widthwise whole area is formed on the proximal end portion of the arm part 114.

Screw holes 116 and 116 for fixation of the leaf spring member 102 to the arm part 114 are formed on one end portion of the leaf spring member mounting part 115 in a widthwise direction thereof.

The leaf spring member 102 is composed of a placing portion 102a arranged within the leaf spring member mounting part 115 and an extension portion 102b which is bent downward at a right angle in a bent portion 119 formed at a rear end portion of the placing portion 102a in the widthwise direction and further bent outward at a right angle, and an engagement projection portion 117 projecting downward in a thickness direction is formed at a distal end portion of the extension portion 102b.

The leaf spring member 102 is fixed to the arm part 114 by inserting screws (not shown) into the screw holes 116 and 116 at the distal end portion of the placing portion 102a in the widthwise direction. Therefore, the leaf spring member 102 is configured such that the extension portion 102a and the extension portion 102b exert a spring function along the thickness direction about regions corresponding to the screw holes 116 and 116 and always bias the engagement projection portion 117 formed at the distal end portion toward the position defining member 87.

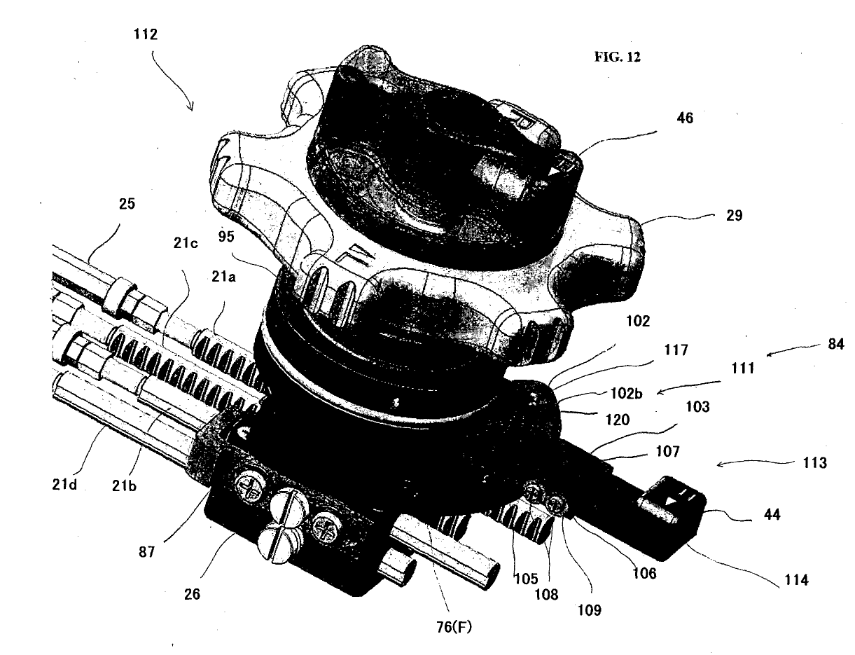

As shown in FIG. 12, the spring constant adjusting member 103 is provided with an adjusting part 107 formed in a flat and approximately-trapezoidal shape and a fixation piece part 106 formed so as to be bent approximately at a right angle along the thickness direction in a side piece portion 104 of the adjusting part 107 and having an adjusting hole 105 composed of a long hole, and the spring constant adjusting member 103 is fixed to a side face portion of the arm part 114 via screws 108 and washers 109 to be movable between the short cylindrical rotation part 95 and the knob part 99 in a lengthwise direction of the arm part 114.

The adjusting part 107 of the spring constant adjusting member 103 is attached to the arm part 98 so as to cover the leaf spring member 102 from the above, and it has an oblique side portion 120 arrange obliquely along a widthwise direction of the leaf spring member 102.

An operation of the endoscope operating apparatus 112 according to this embodiment will be described below.

In this embodiment, when a pressing force of the engagement projection portion 117 to the position defining member 87 is changed by using the leaf spring pressure adjusting mechanism 111, an operator loosens the screws 108 and moves the spring constant adjusting member 103 along the lengthwise direction thereof to change the spring constant of the leaf spring member 102.

In this case, FIG. 11 and FIG. 12 shows a state where the spring constant adjusting member 103 has projected maximally to abut on the rotation part 95, where since the adjusting part 107 is in a state where it covers the placing portion 102a of the leaf spring member 102 most widely, the length sizes of the placing portion 102a and the extension portion 102b exposed from the oblique side portion 120 outward are small.

As a result, in the state shown in FIG. 11 and FIG. 12, in this embodiment, since an effective length size of the leaf spring member 102 functioning as a spring is the shortest, the spring constant becomes the maximum, so that the engagement projection portion 117 is brought into pressure contact with the position defining member 87 most strongly.

Therefore, in this state, when the operator rotationally operates the lock operating handle 113 to cause the engagement projection portion 117 to engage each of the engagement recessed portions 74(L), 75(HL), and 76(F) in order to cause the lock operating part 84 to function, he/she can operate the lock operating handle 113 with strong clicking feeling.

Further, when the operator desires to perform rotational operation of the lock operating handle 113 with softer clicking feeling, he/she loosens the screws 108 and moves the spring constant adjusting member 103 along the adjusting hole 105 in the direction of the knob part 110 by an appropriate size to fix the screws 108 at a proper position.

In this case, since the placing portion 102a is exposed from the oblique side portion 120 further largely, the effective length size of the leaf spring member 102 serving as the leaf spring is made larger, and the spring constant is made small, so that the engagement projection portion 117 is brought into pressure contact with the position defining member 87 more gently.

Therefore, in this state, when the operator rotationally operates the lock operating handle 113 to cause the engagement projection portion 117 to engage each of the engagement recessed portions 74(L), 75(HL), and 76(F) in order to cause the lock operating part 84 to function, he/she can operates the lock operating handle 113 with softer clicking feeling.

Therefore, in the endoscope operating apparatus 112 according to the embodiment, even after the endoscope operating apparatus has been assembled, an operator can conduct adjusting work of the operation load for the lock operation easily and can perform rotational operation of the lock operating part 84 with clicking feeling corresponding to his/her preference.

Incidentally, in the first embodiment and the second embodiment, the case where the lock operating part 46 on the side of the horizontal-direction operating handle is composed of the plunger 57 biased by the coil spring 58 and the cylindrical position defining member 86 provided with the recessed portions 47, 48, and 49 engaged with the distal end portion of the plunger 57, and the lock operating part 84 on the side of the vertical-direction operating handle is composed of the lock operating handle 85 having the arm part 98 and the position defining member 87 formed in a curved plate shape has been described as an example, but the configurations of the lock operating parts are not limited to the above embodiments.

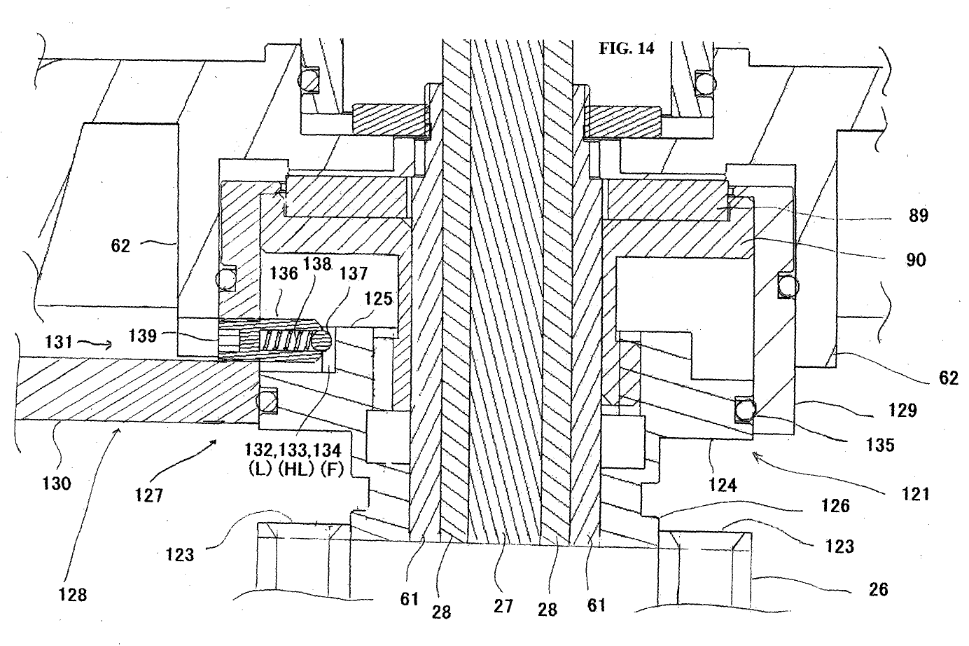

For example, like a third embodiment shown in FIG. 14 to FIG. 16, both lock operating parts of the vertical-direction operating handle 62 may be composed of a position defining member 121 which is wholly formed in an approximately-disc shape and has engagement recessed portions 132(L), 133(HL) and 134(F) similar to the recessed portions 47(L), 48(HL), and 49(F) formed in the position defining member 86 constituting the lock operating part 46 on the side of the horizontal-direction operating handle according to the first embodiment, and a lock operating part 127 providing with a ball 137 which is always biased by a coil spring 138 to be capable of engaging the engagement recessed portions 132(L), 133(HL), and 134(F). Incidentally, same members as those in each of the above embodiments are attached with same reference numeral and explanation thereof is omitted.

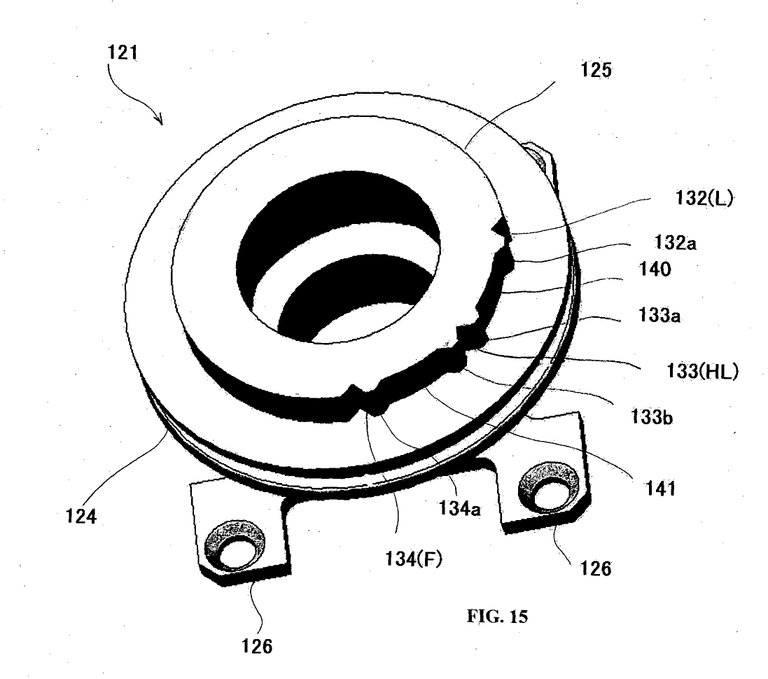

As shown in FIG. 15, the lock operating part 127 according to the third embodiment is composed of a lock operating handle 128 formed in an approximately short cylindrical shape as a whole and provided with a rotation part 129 having an engagement part 131 and an arm part 130 provided so as to protrude from a circumferential face of the rotation part 129 integrally, and a position defining member 121 fixed to an upper face portion of the gear box 26 and arranged in a space within the rotation part 129 in a lower portion of the rotation part 129.

The position defining member 121 is fixed to the upper face portion of the gear box 26 by screws 123 in a state where the center shaft 27, the horizontal-direction drive shaft 28 and the vertical-direction drive shaft 61 have penetrated the position defining member 121.

As shown in FIG. 15, the position defining member 121 is integrally formed as an approximately-disc member wholly, and it is composed of a large-diameter base portion 124, a small-diameter disc-shaped position defining part 125 formed on the large-diameter base portion 124, and fixing portions 126 formed at a lower portion of the large-diameter base portion 124.

Three engagement recessed portions 132(L), 133(HL), and 134(F) indicating a lock position, a half-lock position, and a free position are formed on the position defining part 125 along a circumferential direction thereof like the first embodiment, and the position defining member 121 is arranged in a space of the rotation part 129 such that the rotation part 129 can rotate via an O-ring 135 while abutting on the large-diameter base portion 124.

The engagement part 131 configured so as to be capable of engaging each of the engagement recessed portions 132(L), 133(HL), and 134(F) of the position defining part 125 is provided on the large-diameter base portion 124. The engagement part 131 is provided with a cylindrical body 136 fixed at a lower end portion of a circumferential face portion of the rotation part 129 along a diametrical direction, a ball 137 arranged at a distal end portion of the cylindrical body 136 so as to be capable of advancing and retreating, a coil spring 138 which always biases the ball 137 from the cylindrical body outward, and an adjusting screw 139 arranged at a rear end portion of the cylindrical body 136 and capable of adjusting biasing force of the coil spring 138.

Further, the shape of the arm part 130 integrally formed with the rotation part 124 is the same as that of the arm part 98 in the first embodiment.

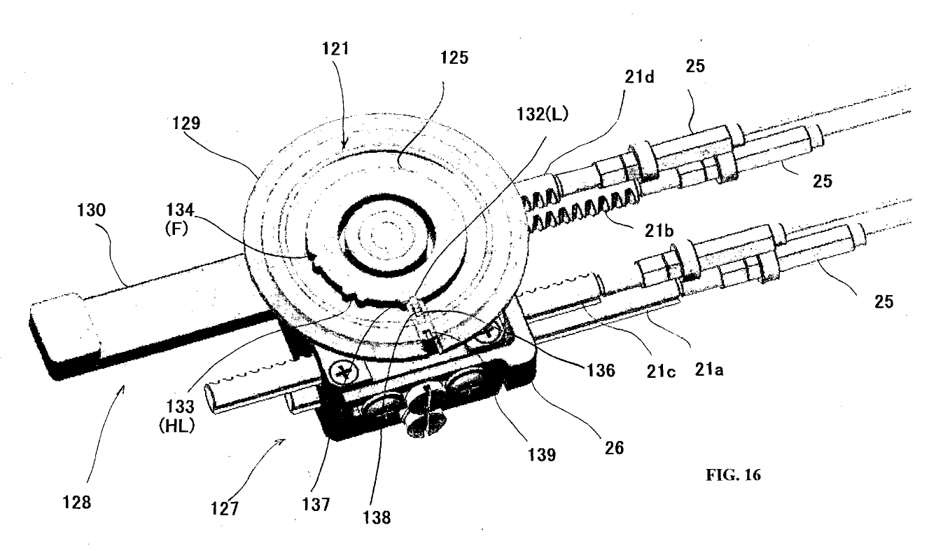

Incidentally, in FIG. 14, a case where the cylindrical body 136 is arranged along a lengthwise direction of the arm part 130 above the arm part 130 is shown, but in FIG. 14, illustration is made in order to conceptually show that the arm part 130 is formed integrally with the rotation part, and the arrangement relationship between the arm part 130 and the cylindrical body 136 lies in arrangement at positions perpendicular to each other, as shown in FIG. 16. FIG. 16 is a sectional view showing a relationship between the position defining member 121 and the lock operating handle 128.

Therefore, in the lock operating part 127 according to this embodiment, when an operator changes the lock state of the vertical-direction operating handle 63 appropriately, he/she rotates the arm part 130 appropriately.

For example, when the vertical-direction operating handle 63 is put in a lock state, as shown in FIG. 16, the ball 137 is disposed in the engagement recessed portion 132(F) by the biasing force of the coil spring 138. When the operator performs transition from the lock state to the half-lock state, he/she rotationally operates the arm part 130 in a clockwise direction in FIG. 16.

In this case, the ball 137 gets over the projection portion 132a forming the engagement recessed portion 132(L) from the lock position where the ball 137 has engaged the engaged recessed portion 132(L) against the biasing force of the coil spring 138 to move outward and reaches the adjacent recessed portion 140.

Thereafter, the ball 137 gets over the projection portion 133a forming the engagement recessed portion 133(HL) forming the half-lock position against the biasing force of the coil spring 138 and reaches the position of the engagement recessed portion 133(HL). Then, when the operator performs change from the half-lock state to the free state, he/she further rotates the arm part 130 in the clockwise direction to cause the ball 137 to get over another projection portion 133b forming the engagement recessed portion 133(HL) and cause the ball 137 to get over the projection portion 134a forming the engagement recessed portion 134(F) forming the free position via the adjacent recessed portion 141, thereby achieving engagement by the biasing force of the coil spring 138.

Therefore, in the lock operating part 127 according to this embodiment, since the position defining member 125 is disposed within the rotation part 129 and it is not protruded outside the apparatus, which is different from the lock operating parts 84 and 127 in the first embodiment and the second embodiment, such an event can be avoided that when an operator performs operation of the endoscope, his/her finger or cloth is carelessly caught by the position defining member, so that the lock state position is changed, which affects examination.

Since the lock operation part is originally a very important mechanism for an operator when he/she operates the bending part, an endoscope operating apparatus where the position defining member is not exposed outside the apparatus and safety of operation can be improved can be provided according to this embodiment.

Incidentally, in this embodiment, the endoscope operating apparatus used in the endoscope as medical equipment inserted into a human body for use has been explained as an example, but a body to be examined is not limited to a human body, it may be a machine, an apparatus, or the like, and the endoscope operating apparatus can be applied to endoscope apparatuses having various applications. Further, respective constituent members constituting the endoscope operating apparatus according to the present invention are not limited to the members described in this text and they can be modified regarding their configurations appropriately within the scope of the present invention described in claims.

### INDUSTRIAL APPLICABILITY

Since the present invention can be widely applied to the endoscope operating apparatus, it has industrial applicability.

### EXPLANATION OF REFERENCE NUMERALS

10 ENDOSCOPE OPERATING APPARATUS
11 ENDOSCOPE APPARATUS MAIN BODY
12 JOINT PART
13 LIGHT GUIDE CABLE CONNECTION PART
14 GAS FEEDING/WATER FEEDLING VALVE
15 BODY
16 VERTICAL-DIRECTION ROTATION OPERATING PART
17 HORIZONTAL-DIRECTION ROTATION OPERATING PART
18 WIRE
18a HORIZONTAL-DIRECTION CONTROL WIRE
18b HORIZONTAL-DIRECTION CONTROL WIRE
18c VERTICAL-DIRECTION CONTROL WIRE
18d VERTICAL-DIRECTION CONTROL WIRE
19 DRIVE PART
20 OPERATING PART
21 RACK
21a HORIZONTAL-DIRECTION DRIVE RACK
21b HORIZONTAL-DIRECTION DRIVE RACK
21c VERTICAL-DIRECTION DRIVE RACK
21d VERTICAL-DIRECTION DRIVE RACK
22 PINION GEAR
22a FIRST PINION GEAR
22b SECOND PINION GEAR
23 ROTATION OPERATING MECHANISM
24 LOCK MECHANISM
25 ANGLE ADJUSTING HOLDER
26 GEAR BOX
28 CENTER SHAFT
28 HORIZONTAL-DIRECTION DRIVE SHAFT
29 HORIZONTAL-DIRECTION OPERATING HANDLE
30 PROJECTION PORTION
31 RECESSED PORTION
32 HORIZONTAL-DIRECTION LOCK MECHANISM
33 BOTTOM FACE PORTION
34 FRICTION PART
35 OPERATING KNOB
36 FRICTION PAD
37 FRICTION PAD SUPPORTING PART
38 OPENING
39 GROOVE PORTION (PRESSURE BONDING PREVENTING PART)
40 PROJECTION PORTION
41 SCREW PORTION
42 SCREW HOLE
43 DISK-SHAPED PART
44 KNOB PART
45 SCREW
46 LOCK OPERATING PART (HORIZONTAL-DIRECTION OPERATING HANDLE SIDE)
47(L) RECESSED PORTION (LOCK POSITION)
48(HL) RECESSED PORTION (HALF-LOCK POSITION)
49(F) RECESSED PORTION (FREE POSITION)
50 ENGAGEMENT PART
51 CYLINDRICAL MEMBER
52 HOLLOW PART
53 CYLINDRICAL SPACE PART
54 HOLE
55 SCREW
56 HORIZONTAL HOLE
57 PLUNGER
58 COIL SPRING
59 LARGE-DIAMETER PART
60 SCREW
61 VERTICAL-DIRECTION DRIVE SHAFT
62 VERTICAL-DIRECTION OPERATING HANDLE
63 PROJECTION PORTION
64 RECESSED PORTION
65 VERTICAL-DIRECTION LOCK MECHANISM
66 LOCK MECHANISM SUPPORTING MEMBER
67 CYLINDRICAL PART
68 LARGE-DIAMETER PART
69 SCREW
70 SUPPORTING PART
73 MOUNTING PART
74(L) ENGAGEMENT RECESSED PORTION (LOCK POSITION)
75(HL) ENGAGEMENT RECESSED PORTION (HALF-LOCK POSITION)
76(F) ENGAGEMENT RECESSED PORTION (FREE POSITION)
77 POSITION PART
78 SIDE EDGE PORTION
79 HOLE
81 GROOVE PORTION
82 GROOVE PORTION
83 SCREW PORTION
84 LOCK OPERATING PART (VERTICAL-DIRECTION OPERATING HANDLE SIDE)
85 LOCK OPERATING HANDLE
86 POSITION DEFINING MEMBER
87 POSITION DEFINING MEMBER
88 SCREW
89 FRICTION PAD
90 FRICTION PAD SUPPORTING MEMBER
91 SHAFT PART
92 SCREW PORTION
93 SUPPORTING PORTION
94 LOWER FACE PORTION
95 ROTATION PART
96 HANDLE PART
97 PROTRUDED RIDGE PORTION
98 ARM PART
99 KNOB PART
100 ENGAGEMENT PROJECTION PORTION
101 LEAF SPRING MEMBER
102 LEAF SPRING MEMBER
102a PLACING PORTION
102b EXTENSION PORTION
103 SPRING CONSTANT ADJUSTING MEMBER
104 SIDE PIECE PORTION
105 ADJUSTING HOLE
106 FIXATION PIECE PART
107 ADJUSTING PART
108 SCREW
109 WASHER
110 KNOB PART
111 LEAF SPRING PRESSURE ADJUSTING MECHANISM
112 ENDOSCOPE OPERATING APPARATUS
113 LOCK OPERATING HANDLE
114 ARM PART
115 LEAF SPRING MEMBER MOUNTING PART
116 SCREW HOLE
117 ENGAGEMENT PROJECTION PORTION
118 WIDTHWISE-DIRECTION REAR END PORTION
119 BENT PORTION
120 OBLIQUE SIDE PORTION
121 POSITION DEFINING MEMBER
123 SCREW
124 LARGE-DIAMETER BASE PORTION
125 POSITION DEFINING PART
126 FIXING PORTION
127 LOCK OPERATING PART
128 LOCK OPERATING HANDLE
129 ROTATION PART
130 ARM PART
131 ENGAGEMENT PART
132(L) ENGAGEMENT RECESSED PORTION (LOCK POSITION)
133(HL) ENGAGEMENT RECESSED PORTION (HALF-LOCK POSITION)
134(F) ENGAGEMENT RECESSED PORTION (FREE POSITION)
135 O-RING
136 CYLINDRICAL BODY
137 BALL
138 COIL SPRING
139 ADJUSTING SCREW
140 RECESSED PORTION
141 RECESSED PORTION
142 COVER MEMBER
143 GROOVE PORTION
144 COVER MEMBER
145 FRICTION PART
146 COVER MEMBER

What is claimed is:

1. An operating apparatus of an endoscope which is provided with a flexible tube inserted into a body to be examined and having a bending part at a distal end thereof and which performs work by bending the bending part according to a manual operation, comprising:
a drive part which can pull a wire which is disposed in the flexible tube, a distal end of the wire being fixed to the bending part and the wire bending the bending part; and an operating part which operates the drive part; wherein the drive part has a rack connected to the wire and a pinion gear meshing with the rack, the operating part is provided with a rotation operating mechanism which can rotationally drive the pinion gear according to manual operation of an operator, and the rotation operating mechanism has a lock mechanism which can impart braking force to an operating part of the rotation operating mechanism,
wherein the wire is provided with two horizontal-direction control wires for performing attitude control of the bending part leftward and rightward, and two vertical-direction control wires for performing attitude control of the bending part upward and downward, racks joined to the four wires, respectively, are provided, a first pinion gear meshing with the horizontal-direction control wires and a second pinion gear meshing with the vertical-direction control wires are provided, a horizontal-direction rotation operating part which can operate the first pinion gear manually and a vertical-direction rotation operating part which can operate the second pinion gear manually are provided, and
wherein the lock mechanism is provided to correspond to each of the horizontal-direction rotation operating part and the vertical-direction rotation operating part.

2. The operating apparatus of an endoscope according to claim 1, wherein the lock mechanism is provided with a friction part which is disposed so as to be capable of being brought into pressure contact with the rotation operating part or being separated therefrom, and a lock operating part which can move the friction part such that the friction part is brought into pressure contact with the rotation operating part or separated therefrom, and
the rotation operating part is composed of an operating handle which rotates along an extension direction of the wire, the friction part is formed of a pad which can be brought into pressure contact with the operating handle or separated therefrom, and the lock operating part is configured so as to move the pad such that the pad is brought into pressure contact with the operating handle or separated therefrom.

3. The operating apparatus of an endoscope according to claim 2, wherein the lock mechanism is provided in the operating handle and the lock operating part is configured to select three positions of a lock position, a half-lock position, and a free position, and the lock operating part is provided with an lock operating handle disposed coaxially with the operating handle to be rotated, a position defining member formed with a position part which can select the three positions according to rotation of the lock operating handle, and an engagement part which can engage the three positions of the position part defining member.

4. The operating apparatus of an endoscope according to claim 3, wherein the position defining member is formed of a cylindrical member arranged and fixed coaxially with the operating handle, recessed portions indicating the lock position, the half-lock position, and the free position are provided on a circumferential face of the cylindrical member along a circumferential direction of the cylindrical member, the operating handle is formed of a knob, and the engagement member is formed of a rod-shaped member provided on the knob and biased toward a rotation center direction of the knob

5. The operating apparatus of an endoscope according to claim 4, wherein the knob is provided with a pressure adjusting part which adjusts biasing force of the rod-shaped member.

6. The operating apparatus of an endoscope according to claim 4, wherein the position defining member is formed of a plate-shaped member arranged and fixed coaxially with the operating handle, recessed portions indicating the lock position, the half-lock position, and the free position are provided on a flat face portion of the plate-shaped member along a rotation direction of the lock operating handle, the lock operating handle is formed as an arm part having a leaf spring part, and the engagement member is formed of a projection portion provided on the leaf spring part.

7. The operating apparatus of an endoscope according to claim 6, wherein the arm part is provided with a pressure adjusting part which can adjust a spring pressure of the leaf spring part.

8. The operating apparatus of an endoscope according to claim 2, wherein the friction part is provided with a friction pad and a support part supporting the friction pad, and a braking face of the friction pad is formed with a pressure bonding preventing part which can prevent a full pressure bonding between the braking face and the rotation operating part.

9. The operating apparatus of an endoscope according to claim 8, wherein the friction pad is formed in a flat disc shape having an opening at a central portion thereof, and the pressure bonding preventing part is composed of a spiral groove portion formed along a circumferential direction of the friction pad.

10. The operating apparatus of an endoscope according to claim 9, wherein the friction pad is made of fluorine-containing rubber.
